(19) United States
(12) Patent Application Publication
(10) Pub. No.: US 2024/0350082 A1
(43) Pub. Date: Oct. 24, 2024

(54) SYSTEM AND METHOD FOR SUBSTANCE ABUSE MONITORING AND MITIGATION

(71) Applicant: Manta MedTech LLC, Newark, DE (US)

(72) Inventor: Kenta ALTEN, Los Altos Hills, CA (US)

(73) Assignee: Manta MedTech LLC, Newark, DE (US)

(21) Appl. No.: 18/667,306

(22) Filed: May 17, 2024

(51) Int. Cl. A61B 5/00; A61B 5/11; A61B 5/145

(52) U.S. Cl. CPC A61B 5/4845; A61B 5/1112; A61B 5/14546; A61B 5/746; A61B 5/747

(57) ABSTRACT

Embodiments are provided that relate to systems comprising a detector component for detecting the presence of a harmful substance in a human body; communication circuitry for transmitting data to a remote device; and a power source configured to supply energy to the detector component and communication circuitry.

### FIELD OF INVENTION

This disclosure relates to systems and processes for substance abuse monitoring and mitigation. The disclosure is more particularly concerned with processes and systems involving sensors and communication for substance abuse monitoring and mitigation.

### BACKGROUND

“As a country, we have a serious substance misuse problem—use of alcohol, illegal drugs, and/or prescribed medications in ways that produce harms to ourselves and those around us. These harms are significant financially with total costs of more than $420 billion annually and more than $120 billion in healthcare (1,2). But these problems are not simply financial burdens—they deteriorate the quality of our health, educational, and social systems, and they are debilitating and killing us—particularly our young through alcohol-related car crashes, drug related violence, and medication overdoses.” [Source: Mclellan A T. Substance Misuse and Substance use Disorders: Why do they Matter in Healthcare? Trans Am Clin Climatol Assoc. 2017; 128:112-130. PMID: 28790493; PMCID: PMC5525418.]

Colorimetric sensing of toxic chemicals, such as chemical warfare agents and narcotics, is of major concern to the Department of Defense community, military, and first responders. In-place chemical sensors, such as M8 or M9 paper and the volatile organic compounds (VOC) test kit, can identify neat toxic chemicals, or more specifically chemical warfare agents, in the liquid phase only. [Source: U.S. Pat. No. 11,402,322B1 published on Aug. 2, 2022]

Opioid overdose is an acute condition resulting from excessive intake of opioids such as morphine, oxycodone, fentanyl, or heroin and was directly implicated in the death of 52,000 Americans in 2016. The sequence of events in death from opioid overdose typically progresses from euphoria to loss of consciousness, respiratory arrest, cardiac arrest and ultimately death. [Source: US20200147307A1 published on May 14, 2020]

The detection of abused drugs in field, which could have a positive impact on reducing the drug traffic is one of the challenges. Scientists are making considerable efforts in solving issues related to selectivity and simultaneous detection from seized samples. Nanomaterials and biomimetic elements have the capacity to improve the selectivity, sensitivity and allowed the simultaneous detection. Additionally, electrochemical sensors could be used in field, and could be equipped with disposable sensors avoiding the contaminations. [Source: Truta F, Florca A, Cernat A, Tertis M, Hosu O, de Wael K, Cristea C. Tackling the Problem of Sensing Commonly Abused Drugs Through Nanomaterials and (Bio) Recognition Approaches. Front Chem. 2020 Nov. 4; 8:561638. doi: 10.3389/fchem.2020.561638. PMID: 33330355; PMCID: PMC7672198.] Therefore, it is of interest to develop new sensing technologies that allow a fast, sensitive, and selective detection of illicit drugs in-the-field.

### SUMMARY

The above-mentioned problems of current systems are addressed by embodiments of the present invention and will be understood by reading and studying the following specification. The following summary is made by way of example and not by way of limitation. It is merely provided to aid the reader in understanding some of the aspects of the invention.

An embodiment relates to a system comprising a detector component for collecting data and detecting presence of a harmful substance in a human body; a first communication circuitry for transmitting the data to a remote device; a first power source configured to supply energy to the detector component and the communication circuitry; and a geofencing component configured to set up a virtual boundary around a defined geographical area.

An embodiment relates to a method comprising sensing presence of a harmful substance in a human body using a utilizing a detector component selected from a group comprising a gas sensor, a chemical sensor, and a biological sensor; transmitting data from the detector component to a remote device via a communication circuitry; and transmitting an emergency alert when at least one of the harmful substance is detected and a wearable device comprising a geofencing application enters or exits the defined geographical area.

An embodiment relates to a method comprising monitoring data and detecting chemical compositions in a subject's body; communicating the data collected by monitoring; transmitting an emergency alert to a remote device when at least one of the harmful substance is detected and a wearable device on the subject's body comprising a geofencing application enters or exits the defined geographical area; and receiving a rescue action from the remote device.

### BRIEF DESCRIPTION OF THE FIGURES

These and other aspects of the present disclosure will now be described in more detail, with reference to the appended drawings showing exemplary embodiments of the present disclosure, in which:

FIG. 1 illustrates a block diagram of an exemplary system in accordance with the present disclosure.

FIG. 2 illustrates a block diagram communication hub of the system, according to one or more embodiments.

FIG. 3 illustrates a block diagram monitoring system within the system, according to one or more embodiments.

FIG. 4 shows use of geofencing for substance abuse prevention, according to one or more embodiments.

### DETAILED DESCRIPTION

### Definitions and General Techniques

For simplicity and clarity of illustration, the drawing figures illustrate the general manner of construction, and descriptions and details of well-known features and techniques may be omitted to avoid unnecessarily obscuring the present disclosure. Additionally, elements in the drawing figures are not necessarily drawn to scale. For example, the dimensions of some of the elements in the figures may be exaggerated relative to other elements to help improve understanding of embodiments of the present disclosure. The same reference numerals in different figures denotes the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements but may include other elements not expressly listed or inherent to such process, method, system, article, device, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the apparatus, methods, and/or articles of manufacture described herein are, for example, capable of operation in other orientations than those illustrated or otherwise described herein.

No element, act, or instruction used herein should be construed as critical or essential unless explicitly described as such. Also, as used herein, the articles “a” and “an” are intended to include items and may be used interchangeably with “one or more.” Furthermore, as used herein, the term “set” is intended to include items (e.g., related items, unrelated items, a combination of related items, and unrelated items, etc.), and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

The terms “couple,” “coupled,” “couples,” “coupling,” and the like should be broadly understood and refer to connecting two or more elements mechanically and/or otherwise. Two or more electrical elements may be electrically coupled together, but not be mechanically or otherwise coupled together. Coupling may be for any length of time, e.g., permanent, or semi-permanent or only for an instant. “Electrical coupling” and the like should be broadly understood and include electrical coupling of all types. The absence of the word “removably,” “removable,” and the like near the word “coupled,” and the like does not mean that the coupling, etc. in question is or is not removable.

As defined herein, two or more elements are “integral” if they are comprised of the same piece of material. As defined herein, two or more elements are “non-integral” if each is comprised of a different piece of material.

As defined herein, “real-time” can, in some embodiments, be defined with respect to operations carried out as soon as practically possible upon occurrence of a triggering event. A triggering event can include receipt of data necessary to execute a task or to otherwise process information. Because of delays inherent in transmission and/or in computing speeds, the term “real time” encompasses operations that occur in “near” real time or somewhat delayed from a triggering event. In a number of embodiments, “real time” can mean real time less a time delay for processing (e.g., determining) and/or transmitting data. The particular time delay can vary depending on the type and/or amount of the data, the processing speeds of the hardware, the transmission capability of the communication hardware, the transmission distance, etc. However, in many embodiments, the time delay can be less than approximately one second, two seconds, five seconds, or ten seconds.

The present disclosure may be embodied in other specific forms without departing from its spirit or characteristics. The described embodiments are to be considered in all respects only as illustrative and not restrictive. The scope of the disclosure is, therefore, indicated by the appended claims rather than by the foregoing description. All changes which come within the meaning and range of equivalency of the claims are to be embraced within their scope.

As defined herein, “approximately” can, in some embodiments, mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

Unless otherwise defined herein, scientific, and technical terms used in connection with the present disclosure shall have meanings that are commonly understood by those of ordinary skill in the art. Further, unless otherwise required by context, singular terms shall include pluralities and plural terms shall include the singular. Generally, nomenclatures used in connection with, and techniques of, health monitoring described herein are those well-known and commonly used in the art.

The methods and techniques of the present disclosure are generally performed according to conventional methods well known in the art and as described in various general and more specific references that are cited and discussed throughout the present specification unless otherwise indicated. The nomenclatures used in connection with, and the procedures and techniques of, embodiments herein, and other related fields described herein are those well-known and commonly used in the art.

The following terms and phrases, unless otherwise indicated, shall be understood to have the following meanings.

The term “narcotics” or “substances” or “harmful substance” refer to drugs that have psychoactive properties and are often associated with pain relief and/or sedation. These substances can be derived from natural sources like plants (such as opium from the poppy plant), semi-synthetic (like heroin, which is derived from morphine), or entirely synthetic (like fentanyl).

The term “detector component” refers to a device within the system that identifies the presence of harmful substances in the human body. It can be a gas sensor, chemical sensor, or biological sensor, each designed to detect different types of harmful materials.

The term “communication circuitry” refers to a part of the system responsible for sending data from the detector component to a remote device. It may include technologies like near field communication (NFC), Bluetooth, or WIFI.

The term “power source” refers to a component of that which provides the necessary energy to operate the detector component and communication circuitry. Typically, this is a battery housed within the system's case.

The term “air flow induction device” refers to a device that is configured to supply consistent air to the detector component, which is crucial for accurate detection of substances.

The term “remote device” refers to a device that receives data from a detector component and/or a monitoring system. It comprises a processor, memory, and its own communication circuitry and power source to process the data.

The term “application” refers to software that resides in the remote device's memory and analyzes the data collected by the monitoring system. This software can be downloaded from an application store and is capable of receiving automatic updates, including new chemical signatures.

The term “adjustable thresholds” refers to a predefined value at which the application can be set to trigger alerts when chemicals are detected in specific concentrations.

The terms “alert” and “notification” refer to a means via which the application can notify the user or broadcast an alert when a chemical is detected.

]

The term “network of devices” refers to a system where multiple devices with monitoring systems share data about detected chemicals to determine their origin and predict how they may spread.

The term “sensing” refers to a process involving the detection of harmful substances using the detector component.

The term “transmitting” refers to a process involving the detection of harmful substances using the detector component. and sending this information to a remote device via communication circuitry.

An embodiment relates to a system comprising a detector component for detecting the presence of a harmful substance in a human body; communication circuitry for transmitting data to a remote device; a power source configured to supply energy to the detector component and communication circuitry.

In an embodiment, the detector component is selected from a group comprising a gas sensor, a chemical sensor, and a biological sensor.

In another embodiment, the communication circuitry includes at least one of a near field communication device, Bluetooth communication device, and WIFI communication device.

In yet another embodiment, the power source includes a battery mounted within a case containing the system.

In yet another embodiment, the system further comprising an air flow induction device to ensure continuous flow of air to the detector component.

In yet another embodiment, the detector component comprises a plurality of sensors capable of detecting biological materials, narcotics, and combinations thereof.

In yet another embodiment, the remote device includes a processor, memory, communication circuitry, and a power source for processing the data collected by the monitoring system.

In yet another embodiment, system further comprising software stored in the memory of the remote device for analyzing the data collected by the monitoring system.

In yet another embodiment, the software is an application downloadable from an application store and configured to be automatically updated with new chemical signatures.

In yet another embodiment, the application includes adjustable thresholds for detecting chemicals present in certain amounts.

In yet another embodiment, the application is configured to broadcast an alert or generate a notification upon detection of a chemical.

In yet another embodiment, the application uses location information from the remote device to identify the location of the human body. An application can use the remote device's location data to pinpoint where a chemical has been detected.

In yet another embodiment, the application is configured to alert authorities in the event certain chemicals are detected.

In yet another embodiment, a network of devices with monitoring systems is configured to share data regarding the dispersion of detected chemicals to calculate a point of origin and predict dispersion patterns.

An embodiment relates to a method comprising sensing presence of a harmful substance in a human body using a detector component selected from a group comprising a gas sensor, a chemical sensor, and a biological sensor; and transmitting data from the monitor component to a remote device via a communication circuitry.

An embodiment relates to a method comprising monitoring and detecting chemical compositions in a subject's body; communicating the data collected by monitoring; and detecting to a remote device; and receiving a rescue action from the remote device.

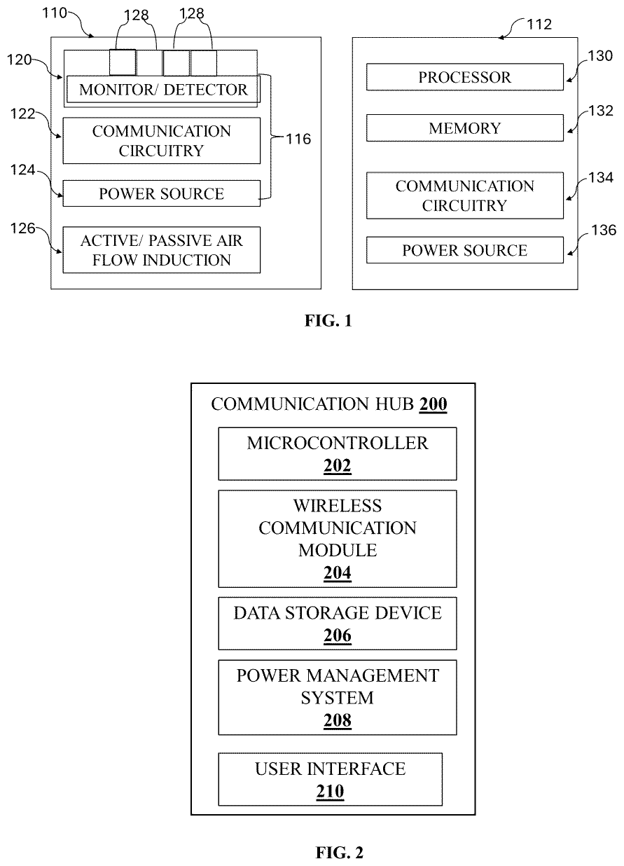

Referring to FIG. 1, it illustrates a block diagram of an exemplary system in accordance with the present disclosure. The monitoring system 116 generally comprises a monitor/detector component 120. One monitor/detector component that is particularly well-suited for purposes of the present disclosure is set forth in U.S. Pat. No. 8,629,770 to Hummer et al. and U.S. Pat. No. 7,176,793 to Hummer, both of which are incorporated herein by reference in their entireties. Other types of monitor/detector components can also be used in accordance with the present disclosure.

The monitoring system further comprises communication circuitry 122 and a power source 124. The communication circuitry 122, in one embodiment, comprises at least one of a near field communication device, Bluetooth communication device, WIFI communication device, or any other suitable communication circuitry for establishing communications with the remote device 112. The power source 124 can be a power supply such as a battery (lithium or other) mounted or otherwise contained within case 110. In other embodiments, the power source 124 can be an antenna configured to receive energy wirelessly and supply the received energy to one or both of the monitor/detector component 120 and/or communication circuitry 122 such that no onboard battery is required for operation of the monitoring system 116. In still other arrangements, the power source 124 can be a connector configured to couple with a port of the remote device 112 to receive power from a second power source 136 of the remote device 112.

An active or passive air flow induction device 126 can be provided for ensuring adequate and or continuous flow of air to the monitor/detector 120. Such devices can comprise fans, micropumps, louvers, vents etc. An active induction device can be separately replaceable within the system and can comprise its own power supply. Alternatively, an active induction device can be configured to receive power from power source 124.

It should be appreciated that the monitor/detector component 120 can comprise a plurality of sensors 128. The sensors 128 can be individually replaceable or can be replaced as a unit. Replacement of the sensors may be necessary due to sensor degradation. In other situations, a user may wish to detect certain chemicals and will choose which sensors to install in the system.

In one embodiment, the entire monitoring system 116 is replaceable as a unit.

The sensors 128 may detect harmful materials, such as biological materials, narcotics and other illegal drugs, or combinations thereof.

It will be appreciated that the monitoring system 116 is configured to communicate with the remote device 112. That is, the monitoring system 116 collects data and transmits or otherwise shares the collected data with the remote device 112 for processing. The remote device 112 of the illustrated embodiment comprises a processor 130, a memory 132, a second communication circuitry 134, and a second power source 136. It will be appreciated that the remote device 112 can comprise a wide variety of additional components as is conventional. Such additional components can comprise a display device, input device, various sensors, various antennas, etc.

Data collected by the monitor/detector 120 is transmitted via first communication circuitry 122 to second communication circuitry 134 of the remote device 112. Other data, such as sensor state, status, performance data, and the like can also be transmitted to the remote device 112. Any suitable manner of transmitting the data from the monitoring system 116 to the remote device 112 can be employed.

The data collected and transmitted by the monitoring system 116 is then processed by the phone to detect one or more chemicals in accordance with one or more methods set forth in U.S. Pat. No. 8,629,770 to Hummer et al. and U.S. Pat. No. 7,176,793 to Hummer. To this end, suitable software for analyzing the data is stored in memory 132 of the remote device 112. Other detection and/or analyzing methods and techniques may also be used in conjunction with aspects of the present disclosure.

The system comprises a server component; and a mobile application component wirelessly coupled to the server component; wherein the server component and the mobile application component are collectively operable for, in an interactive manner, receiving input from a user and presenting the user with output based on the input received from the user, wherein the input received from the user comprises one or more of personal information, activity information, and activity related to alcohol and/or drug use, and wherein the output presented to the user comprises one or more of coaching information and counseling information

In one embodiment, the software stored in memory 132 of the remote device 112 can be in the form of an application, or “app”, that is downloaded from an application store or the like. The application can be provided with various “signatures” of chemicals. The signatures can be compared to the data to determine whether the chemical signature was detected by the monitoring system 116. The application can be configured to be automatically updated with new signatures as the need to detect particular chemicals arises. That is, it is possible to provide new and/or additional chemical signatures for the application to check against the data to detect specific chemicals.

The application further comprises features such as adjustable thresholds. For example, for some chemicals that are routinely present in certain amounts and/or not generally considered dangerous below certain levels, the application can be configured to detect or trigger an alarm when a threshold amount is met or exceeded. For some chemicals which are considered dangerous in any amount, the thresholds can be fixed and.

The application can be further configured to, once a chemical is detected, share the detection information. For example, the application can be configured to use the second communication circuitry 134 to broadcast an alert (or generate a notification) via any suitable communications network (e.g., WIFI, NFC, Bluetooth, cell, etc.). The alert may be directly sent to other remote devices and/or personal communication devices in the area or may be sent to a server, or through a network and then on to devices within a range of a given location. Accordingly, the application can be configured to use location information from a GPS chip, WIFI or any other location information available to the remote device 112 to identify the location of the human body.

The application can be configured to alert the authorities in the event certain chemicals are detected. For example, the detection of any amount of harmful substance can trigger information relating to the location, time, etc. of the detection to be forwarded to certain designated authorities for threat management/mitigation.

It should be appreciated that a network of devices having monitoring systems, each detecting a certain chemical, can be configured to share valuable data regarding the dispersion of the particular chemical. For example, devices in close proximity to each other and the point of origin of the chemical may detect a greater concentration of the chemical than devices further away from the point of origin. Using this data and an appropriate dispersion model, a point of origin can be calculated. This can allow responsive action to be taken more quickly than otherwise would be the case.

Similarly, the data (location, concentration, etc.) from a plurality of such devices can be used to predict dispersion of the chemical so that preemptive action can be taken to minimize exposure of humans to the detected chemical.

Providing the monitoring system 116 in a separate component that is attachable to a phone or other personal communication device has several advantages. For example, any and all such devices can become monitors/detectors upon the provision of a suitable case or other component. Accordingly, a consumer can decide whether to add the functionality. In addition, the orientation, location, and other aspects of the positioning of the sensor elements within the case or other component can be standardized to provide more consistent detection as compared to placing the sensor elements within various different models of remote devices. This is because the myriad phone manufacturers and models each have different space constraints that would dictate different available locations, orientations, etc. for the sensor elements within the phone. As such, some sensor elements would be in a better position within a respective phone to detect chemicals than other phones. This can lead to widely varying detection accuracy between different phones exposed to the same concentration of a given chemical.

It should be appreciated that, although the monitoring system 116 is illustrated as part of a case 110, the monitoring system can also be provided as a separate unit attachable either directly to a remote device or the like, or attachable to a case in which a remote device is contained.

In an embodiment the communication circuitry comprises a communication hub that is in communication with the monitoring system and a remote device. Embodiments of the present invention provide a wearable intelligent communication hub that handles the complexity of underlying communication channels and sensors while providing an easy-to-use plug-and-play interface that lets first responders focus on their job. In embodiments, a communication hub seamlessly establishes communication links with different types of communication systems to create a communication network where information can be passed to and from the communication hub. Moreover, embodiments of the communication hub gather sensor information and pass the gathered sensor data through the established communication links that make up the communication network. In some embodiments, the communication hub enacts routing instructions that create a communication path through communication links to command or emergency operation centers as well as other teammates in the same response operation.

In one embodiment, a communication hub that manages communication signals between the communication hub and a plurality of communication systems that use a plurality of different communication signal formats to form a communication network is provided. The communication hub also manages sensor signals between the communication hub and a plurality of sensors that use a plurality of different sensor signal formats. The communication hub includes communication signal gateways, sensor signal gateways, a controller, a storage medium and a power source. The communication signal gateways are configured to receive and transmit the communication signals with the plurality of different communication signal formats. The sensor signal gateways in turn are configured to receive the sensor signals with the plurality of different sensor signal formats. The controller is configured to dynamically interface the different communication signal formats of the received communication signals at the communication signal gateways with a communication hub signal format used by the communication hub to establish communication links with the communication systems associated with the received communication signals. The controller is further configured to dynamically interface the different sensor signal formats of the received sensor signals at the sensor signal gateways into the communication hub signal format to gather sensor information. The controller is further configured to establish at least one routing path to a destination hub using at least one of the established communication links. The controller further yet is configured to communicate the sensor information through the at least one established routing path. The storage medium is in communication with the controller. The storage medium is configured to store operating functions of the controller. The power source is selectively coupled to provide power for the communication hub.

In an embodiment, the controller is further configured to implement communication manager, configuration manager and sensor manager functions stored in the storage medium. The communication manager function is configured to establish the communication links through the communication signal gateways. The configuration manager function is configured to establish the at least one established routing path to the destination hub using at least one of the established communication links. The sensor manager function is configured to dynamically interface the different sensor signal formats of the sensor signals received at the sensor signal gateways into a communication hub sensor signal format used by the communication hub and to communicate the sensor information through the at least one established routing path.

In an embodiment, the communication signal gateways further comprise a plurality of communication receivers and a plurality of communication transmitters. The plurality of communication receivers comprises at least one first communication receiver that is configured to receive a first type of communication signal and at least one second communication receiver that is configured to receive a second type of communication signal. The plurality of communication transmitters comprises at least one first communication transmitter that is configured to transmit the first type of communication signal and at least one second communication transmitter that is configured to transmit the second type of communication signal.

In an embodiment, the controller is further configured to monitor for the different types of communication signals received at the communication signal gateways.

In an embodiment, the communication hub further includes a power on indicator and an established communication link indicator. The power on indicator is selectively coupled to the power source and is configured to indicate when the communication hub is operational. The established communication link indicator is in communication the controller. The established communication link indicator is configured to indicate when at least one established communication link has been established. Embodiments of the present invention provide a wearable intelligent communication hub that handles the complexity of underlying communication channels and sensors while providing an easy-to-use plug-and-play interface that lets first responders focus on their job. In embodiments, a communication hub seamlessly establishes communication links with different types of communication systems to create a communication network where information can be passed to and from the communication hub. Moreover, embodiments of the communication hub gather sensor information and pass the gathered sensor data through the established communication links that make up the communication network. In some embodiments, the communication hub enacts routing instructions that create a communication path through communication links to command or emergency operation centers as well as other teammates in the same response operation.

In one embodiment, a communication hub that manages communication signals between the communication hub and a plurality of communication systems that use a plurality of different communication signal formats to form a communication network is provided. The communication hub also manages sensor signals between the communication hub and a plurality of sensors that use a plurality of different sensor signal formats. The communication hub includes communication signal gateways, sensor signal gateways, a controller, a storage medium and a power source. The communication signal gateways are configured to receive and transmit the communication signals with the plurality of different communication signal formats. The sensor signal gateways in turn are configured to receive the sensor signals with the plurality of different sensor signal formats. The controller is configured to dynamically interface the different communication signal formats of the received communication signals at the communication signal gateways with a communication hub signal format used by the communication hub to establish communication links with the communication systems associated with the received communication signals. The controller is further configured to dynamically interface the different sensor signal formats of the received sensor signals at the sensor signal gateways into the communication hub signal format to gather sensor information. The controller is further configured to establish at least one routing path to a destination hub using at least one of the established communication links. The controller further yet is configured to communicate the sensor information through the at least one established routing path. The storage medium is in communication with the controller. The storage medium is configured to store operating functions of the controller. The power source is selectively coupled to provide power for the communication hub.

In an embodiment, the controller is further configured to implement communication manager, configuration manager and sensor manager functions stored in the storage medium. The communication manager function is configured to establish the communication links through the communication signal gateways. The configuration manager function is configured to establish the at least one established routing path to the destination hub using at least one of the established communication links. The sensor manager function is configured to dynamically interface the different sensor signal formats of the sensor signals received at the sensor signal gateways into a communication hub sensor signal format used by the communication hub and to communicate the sensor information through the at least one established routing path.

In an embodiment, the communication signal gateways further comprise a plurality of communication receivers and a plurality of communication transmitters. The plurality of communication receivers comprises at least one first communication receiver that is configured to receive a first type of communication signal and at least one second communication receiver that is configured to receive a second type of communication signal. The plurality of communication transmitters comprises at least one first communication transmitter that is configured to transmit the first type of communication signal and at least one second communication transmitter that is configured to transmit the second type of communication signal.

In an embodiment, the controller is further configured to monitor for the different types of communication signals received at the communication signal gateways.

In an embodiment, the communication hub further includes a power on indicator and an established communication link indicator. The power on indicator is selectively coupled to the power source and is configured to indicate when the communication hub is operational. The established communication link indicator is in communication the controller. The established communication link indicator is configured to indicate when at least one established communication link has been established.

Referring to FIG. 2, the communication hub 200 comprises a first microcontroller 202, a wireless communication module 204, a data storage device 206, a power management system 208, and a user interface 210. The microcontroller 202 controls the operation of the communication hub, manages data transmission, and interfaces with other components. The wireless communication module 204 facilitates communication with monitoring system and external systems via secure wireless protocols such as Bluetooth Low Energy (BLE) or Wi-Fi. The data storage device 206 securely stores data transmitted from monitoring devices for later analysis and retrieval. The power management system 208 regulates power supply to ensure uninterrupted operation. The user interface 210 provides status indicators and interaction options for users.

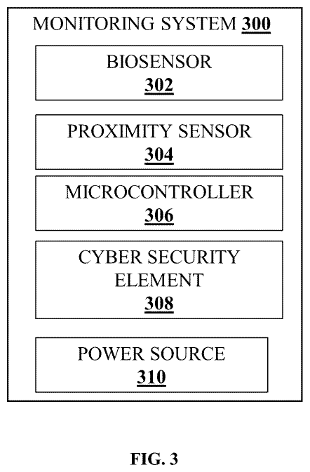

Referring to FIG. 3, it shows a monitoring system, according to one or more embodiments. The monitoring system 300 comprises a biosensor 302, a proximity sensor 304, a microcontroller 306, a cyber security element 308, and a power source 301. The biosensor 302 detects and measures drug concentrations within the subject's body through biological samples (e.g., blood or saliva).

The proximity sensor 304 determines the distance between the subject's body and the communication hub. The microcontroller 306 controls sensor data acquisition, processing, and communication with the communication hub. The cyber security element 308 encrypts sensitive data before transmission to ensure privacy and prevent tampering. The power source 310 comprises a rechargeable battery to enable continuous operation.

The monitoring system continuously samples biological fluids to measure drug concentrations using integrated biosensors. Data collected by the biosensors are processed by the microcontroller and encrypted by the cyber security element to prevent unauthorized access. Encrypted data packets are transmitted wirelessly to the communication hub at regular intervals. The communication hub receives and stores the data for further analysis or transmission to external systems, such as healthcare professionals' dashboards or electronic medical records.

The monitoring system utilizes a proximity sensor to measure the distance between the subject's body and the communication hub. Distance data are processed internally and transmitted securely to the communication hub along with drug concentration data. The communication hub stores proximity data and can trigger alerts if the subject moves beyond predefined proximity thresholds.

All data transmitted between the monitoring system and the communication hub are encrypted using robust encryption algorithms to prevent interception and tampering. Data stored within the communication hub are encrypted and stored securely to prevent unauthorized access. Access to sensitive data and device settings is restricted through user authentication mechanisms, such as passwords or biometric verification. Personally identifiable information is anonymized before transmission or storage to protect the subject's privacy. Firmware and software updates are regularly deployed to address security vulnerabilities and enhance overall system security.

Both the communication hub and monitoring system are housed in tamper-resistant enclosures designed to deter unauthorized physical access. Internal sensors detect attempts to tamper with the devices, triggering alerts and initiating protective measures such as data wipe or disabling functionality. Access to critical components or ports is restricted to using physical locks or seals, requiring authorized personnel for maintenance or servicing.

In an embodiment, the internal sensors are connected via wireless sensor networks (WSNs). The WSNs comprise one or more sensor nodes (also known as motes). These nodes are spatially distributed autonomous devices that can receive input information from connected sensors, process the data, and transmit the output to other devices via a wireless network.

Optionally, the system further comprises a testing device coupled to the mobile application component and adjusting one or more of the coaching and counseling information based on results obtained from the testing device.

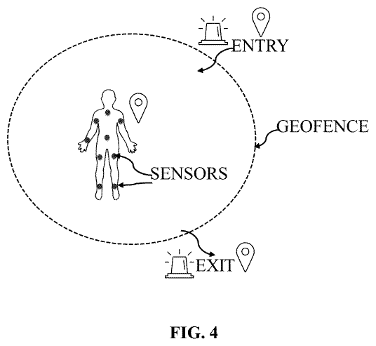

Referring to FIG. 4, it shows use of geofencing for substance abuse prevention, according to one or more embodiments. Geofencing can create virtual boundaries around Global Positioning Systems (GPS) coordinates in order to detect a user's presence in a specific area of interest, e.g. bars, clubs, liquor stores- and GPS tracking allow for real-time study of user behavior, as well as enhanced ability to influence it.

A method and apparatus for determining a baseline of one or more of biological, chemical, and physiological characteristics of a subject is provided. The one or more of biological, chemical, and physiological characteristics of a subject and context information associated with the subject are obtained. Detecting a time period that a subject is in a baseline state based on the acquired one or more physiological characteristics of the subject and the context information associated with the subject. Determining the baseline for the one or more physiological characteristics of the subject from values of at least one of the acquired one or more physiological characteristics in the detected time period.

Consciousness or wakefulness of the user is monitored by requiring the user to engage or activate actuator(s) specifically designed to require conscious effort. Involuntary disengagement or release of actuator(s) suggests impaired consciousness and results in an alarm. Should the user fail to respond to this alarm by re-engaging the actuator(s), preloaded naloxone is injected automatically to rescue the unconscious user.

Impaired consciousness can also be detected by failure of user to respond to a series of prompts at timed intervals. The actuator may be a motion detection sensor such as an accelerometer or video camera or may utilize facial recognition technology.

Monitoring impaired consciousness as a surrogate for opioid overdose is distinct from monitoring of vital signs, which aims to detect respiratory and cardiac depression or arrest. Monitoring impaired consciousness is strategic as it occurs earlier in the cascade of symptoms associated with opioid overdose, thus providing precious additional time for rescue treatment to take effect. Furthermore, this method alleviates the cost, regulatory requirements, and technical complexity of ensuring accurate measurement of vital signs and setting appropriate parameters.

The present invention also describes processes for films incorporating the self-indicating colorimetric response materials; wearable self-indicating colorimetric response chemical sensors; solid, liquid, and vapor chemical detection via real-time colorimetric response; and/or toxic chemical detection devices, such as optical, spectral, and/or electronic chemical sensors.

In some embodiments, the system comprises a continuous monitor to measure a subject's oxygenation and/or respiration coupled to a device configured to stimulate the subject's respiratory drive and/or summon medical assistance. In some embodiments, there are provided systems, devices, and methods to assist in preventing subjects from overdosing themselves with narcotics.

In some embodiments, the system may comprise a commercially available pulse oximeter and/or a respiratory monitor to continuously monitor a subject's level of oxygenation/respiration.

In an embodiment, the system comprises an apparatus that automatically delivers a narcotic reversal agent to restore normal breathing. The apparatus minimizes the delay in rescuing subjects with abnormal monitored alarm conditions otherwise at risk of death, irreversible brain damage, or other potentially avoidable outcomes.

### INCORPORATION BY REFERENCE

All references, including granted patents and patent application publications, referred herein are incorporated herein by reference in their entirety.
U.S. Pat. No. 11,402,322B1 titled “Self-indicating colorimetric response materials for removal and sensing of toxic chemicals and narcotics”;
US20200147307A1 titled “Systems and method for detection and treatment of opioid overdose”;
U.S. Pat. No. 8,629,770B2 titled “Sensor for container monitoring system”;
U.S. Pat. No. 7,176,793B1 titled “Container monitoring device”.

What is claimed is:

1. A system comprising:
a detector component for collecting data and detecting presence of a harmful substance in a human body;
a first communication circuitry for transmitting the data to a remote device;
a first power source configured to supply energy to the detector component and the communication circuitry; and
a geofencing component configured to set up a virtual boundary around a defined geographical area.

2. The system of claim 1, wherein the detector component is selected from a group comprising a gas sensor, a chemical sensor, and a biological sensor.

3. The system of claim 1, wherein the communication circuitry includes at least one of a near field communication device, Bluetooth communication device, and WIFI communication device.

4. The system of claim 1, wherein the power source includes a battery mounted within a case containing the system.

5. The system of claim 1, further comprising an air flow induction device to ensure continuous flow of air to the detector component.

6. The system of claim 1, wherein the detector component comprises a plurality of sensors capable of detecting biological materials, narcotics, and combinations thereof.

7. The system of claim 1, wherein the remote device includes a processor, a memory, a second communication circuitry, and a second power source for processing the data collected by the detector component.

8. The system of claim 7, further comprising software stored in the memory of the remote device for analyzing the data collected by the detector component.

9. The system of claim 8, wherein the software is an application downloadable from an application store and configured to be automatically updated with new chemical signatures.

10. The system of claim 9, wherein the application comprises adjustable thresholds for detecting a chemical present in certain amounts.

11. The system of claim 10, wherein the application is configured to broadcast an alert or generate a notification upon detection of the chemical.

12. The system of claim 11, wherein the application uses location information from the remote device to identify a location of the human body.

13. The system of claim 9, wherein the application is configured to alert authorities in event certain chemicals are detected.

14. The system of claim 9, wherein a network of devices with monitoring systems is configured to share the data regarding dispersion of the chemical detected to calculate a point of origin and predict dispersion patterns.

15. The system of claim 9, wherein the system is configured to transmit an emergency alert comprising an emergency location when a wearable device comprising a geofencing application device enters or exits the defined geographical area.

16. A method comprising:
sensing presence of a harmful substance in a human body using a utilizing a detector component selected from a group comprising a gas sensor, a chemical sensor, and a biological sensor;
transmitting data from the detector component to a remote device via a communication circuitry; and
transmitting an emergency alert when at least one of the harmful substance is detected and a wearable device comprising a geofencing application enters or exits the defined geographical area.

17. The method of claim 16, wherein the emergency alert comprises an emergency location.

18. A method comprising:
monitoring data and detecting chemical compositions in a subject's body;
communicating the data collected by monitoring;
transmitting an emergency alert to a remote device when at least one of the harmful substance is detected and a wearable device on the subject's body comprising a geofencing application enters or exits the defined geographical area; and
receiving a rescue action from the remote device.
